(19) United States
(12) Patent Application Publication
(10) Pub. No.: US 2012/0041076 A1
(43) Pub. Date: Feb. 16, 2012

(54) GEL FORMING SOLUTION AND METHOD FOR PRODUCING GEL

(75) Inventor: Kei HIRUMA, Chino-shi (JP)

(73) Assignee: SEIKO EPSON CORPORATION, Tokyo (JP)

(21) Appl. No.: 13/207,640

(22) Filed: Aug. 11, 2011

(30) Foreign Application Priority Data
Aug. 11, 2010 (JP) 2010-180107

(51) Int. Cl. A61K 47/10; A61K 47/36; A61K 8/73; C12N 9/00; A23L 1/0532; B01J 31/02; C09K 3/00; A61K 6/00; A23L 1/05; A61K 8/34; C12N 11/04

(52) U.S. Cl. 514/769; 514/772; 435/183; 435/177; 502/172; 106/499; 252/182.12; 106/35; 426/573; 426/575; 426/61

(57) ABSTRACT

A method for producing a gel includes a liquid droplet ejection step in which a first liquid is ejected in the form of a droplet to a second liquid to allow the first liquid and the second liquid to react with each other, thereby producing a gel. The first liquid is a gel forming solution containing an alcohol-based solvent, wherein the alcohol-based solvent is contained therein in an amount of 4% or more by weight and less than 70% by weight of the total amount of the gel forming solution.

The present application claims priority to Japanese Patent Application 2010-180107 filed on Aug. 11, 2010, which is incorporated by reference in its entirety.

### BACKGROUND

1. Technical Field

Embodiments of the present invention relate to a gel forming solution and a method for producing a gel.

2. Related Art

A gel can be produced by allowing a first solution and a second solution to react with each other. For example, an inkjet method can be used to produce a gel by ejecting one of two kinds of liquids, which liquids form a gel when coming into contact with each other, into the other liquid (see e.g., JP-A-2007-111591). As a material composition with which a gel bead and also a three-dimensional structure are produced by employing an inkjet technique and materials with respect to the resulting product, several kinds of materials which gelate are described in JP-A-2007-111591, and an inkjet technique is used in any of the production methods described therein.

A gel is used in a wide range of fields, including, for example, copying paper, pharmaceutical products, agricultural chemicals, regenerative medicine and the like. Various methods for producing a gel, such as an interfacial polymerization method and a submerged curing coating method, can be employed.

However, there are problems in conventional methods. Usable materials, for example, are limited by the respective production methods. Further, the shape, size, and strength of a gel produced by any method are not uniform. Accordingly, there is a need for a method in which various materials can be used and with which the shape, size, and strength of a gel can be controlled with high accuracy.

In JP-A-2007-111591, an aqueous solution of sodium alginate is ejected by an inkjet method. However, water is serving as a main solvent in the aqueous solution and is liable to dry. When an inkjet head is filled with the aqueous solution, the aqueous solution is sometimes exposed to air at the tip of the nozzle for a long period of time. Therefore, there is a possibility that oxidative deterioration of the aqueous solution, evaporation thereof, or the like, may cause the nozzle to clog. The performance of intermittent ejection is lowered. In other words, nozzles that eject the aqueous solution intermittently may clog during the time that the nozzles are not ejecting the solution. As a result, the aqueous solution can no longer be ejected.

### SUMMARY

An advantage of some aspects of the invention is to solve at least a part of the problem described above, and can be realized as the following embodiments or application examples.

### Application Example 1

According to Application Example 1 of the invention, a gel forming solution containing an alcohol-based solvent is provided. The alcohol-based solvent is contained therein in an amount of 4% or more by weight and less than 70% by weight of the total amount of the gel forming solution. In other words, the alcohol-based solvent makes up between 4% and 70% of the total amount of the gel forming solution by weight.

As a result of intensive studies by experiments, the present inventors found that by adjusting an amount of an alcohol-based solvent in a gel forming solution, a strength of a gel can be controlled. That is, if the alcohol-based solvent is contained in a gel forming solution in an amount of 4% or more by weight and less than 70% by weight of the total amount of the gel forming solution, the strength of the resulting gel can be increased compared with the case where an alcohol-based solvent is not contained in a gel forming solution or with a case where an alcohol-based solvent is contained therein in an amount of less than 4% by weight or 70% or more by weight of the total amount of the gel forming solution. Therefore, by adjusting the amount of an alcohol-based solvent to be added to a gel forming solution, a gel with a desired strength can be produced.

The alcohol-based solvent exhibits a high water retaining property. As a result, the saturated water content of the gel forming solution can be increased so as to cause less water to be released even if the environment changes. Therefore, by filling an ink flow path or a nozzle of an inkjet head with this gel forming solution, problems such as failure of the gel forming solution to eject and deterioration of the gel forming solution can be solved when the solution is ejected from the inkjet head and/or when the solution is ejected intermittently.

### Application Example 2

According to Application Example 2 of the invention, in the gel forming solution of the application example of the invention described above, the alcohol-based solvent is at least one solvent selected from a group consisting of polyhydric alcohols.

According to the configuration of Application Example 2 of the invention, by using at least one solvent selected from the group consisting of polyhydric alcohols, the alcohol-based solvent can be easily used.

### Application Example 3

According to Application Example 3 of the invention, in the gel forming solution of the application example of the invention described above, the alcohol-based solvent is contained in an amount of 40% or more by weight and less than 60% by weight of the total amount of the gel forming solution.

According to the configuration of Application Example 3 of the invention, by incorporating a specific alcohol-based solvent in the gel forming solution in a predetermined amount, a gel having a desired strength can be formed.

### Application Example 4

According to Application Example 4 of the invention, a method for producing a gel is provided. The method includes ejecting a first liquid in the form of a droplet to a second liquid to allow the first liquid and the second liquid to react with each other, thereby producing a gel. The first liquid is a gel forming solution containing an alcohol-based solvent in an amount of 4% or more by weight and less than 70% by weight of the total amount of the gel forming solution.

According to Application Example 4 of the invention, by incorporating a specific alcohol-based solvent in the gel forming solution in a predetermined amount, the strength of the gel can be controlled.

Since the gel forming solution containing a specific alcohol-based solvent in a predetermined amount is used, a high water retaining property of the alcohol-based solvent is exhibited. The saturated water content of the gel forming solution can be increased so as to cause less water to be released even if the environment changes. Therefore, by filling an ink flow path or a nozzle of an inkjet head with this gel forming solution, problems such as failure of the gel forming solution to eject and deterioration of the gel forming solution can be solved when the solution is ejected from the inkjet head and/or when the solution is ejected intermittently.

### Application Example 5

According to Application Example 5 of the invention, in the method for producing a gel of the application example of the invention described above, at least the first liquid is preferably ejected in the form of a droplet in the liquid droplet ejection step.

According to the configuration of Application Example 5 of the invention, for example, filling an ink flow path or a nozzle of an inkjet head with this gel forming solution solves problems such as failure of the gel forming solution to eject from the nozzle and deterioration of the gel forming solution when the solution is ejected from the inkjet head and/or when the solution is ejected intermittently.

### BRIEF DESCRIPTION OF THE DRAWINGS

The invention will be described with reference to the accompanying drawings, wherein like numbers reference like elements.

FIGS. 1A and 1B are schematic cross-sectional views showing a gel according to an embodiment of the invention.

FIG. 2 is a flow chart showing a method for producing a gel according to an embodiment of the invention.

FIGS. 3A and 3B are schematic process charts showing a method for producing a gel according to an embodiment of the invention.

FIG. 4 is a graph showing a relationship between a gel strength and an amount of glycerin added to a gel forming solution according to an embodiment of the invention.

FIG. 5 is a table showing a relationship of a strength (load) and a gel condition with respect to an amount of glycerin added to a gel forming solution according to an embodiment of the invention.

FIG. 6 is a graph showing a relationship between an intermittent time (e.g., a time between ejections of a gel forming solution from nozzle) and a percentage of ejecting nozzles in response to a change in an amount of glycerin added to a gel forming solution according to an embodiment of the invention.

FIG. 7 is a table showing a relationship between an amount of glycerin added to a gel forming solution according to an embodiment of the invention and performance of intermittent ejection.

### DESCRIPTION OF EXEMPLARY EMBODIMENTS

Hereinafter, specific embodiments of a gel forming solution and a method for producing a gel will be described with reference to the accompanying drawings. In the method for producing a gel according to an embodiment of the invention, a liquid is ejected by an inkjet method to allow the liquid to gelate.

First, a gel to be formed by the method for producing a gel according to an embodiment of the invention will be described.

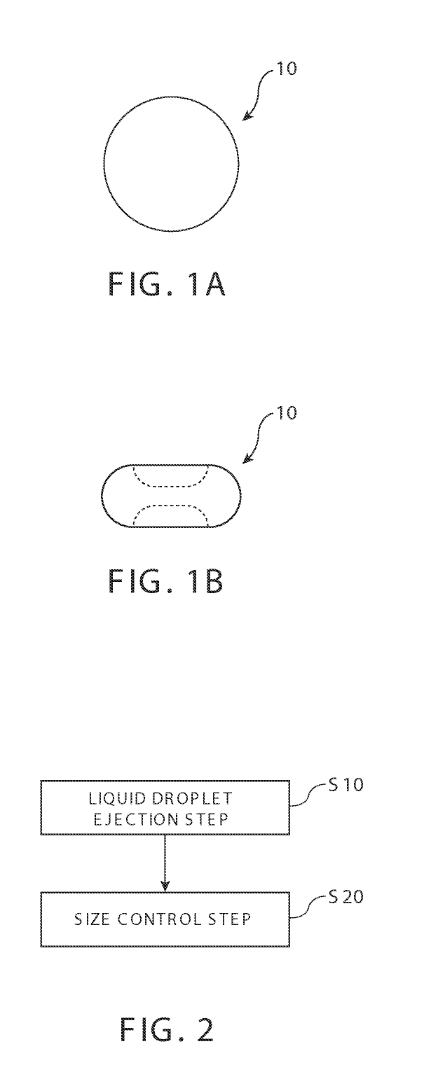

FIGS. 1A and 1B are schematic cross-sectional views showing a gel according to this embodiment. As shown in FIG. 1A, a gel 10 may have a flexible spherical shape. Alternatively, as shown in FIG. 1B, the gel 10 may have a flexible disk shape with dents as indicated by the dashed lines in the center. The gel 10 in FIG. 1B, for example, has a shape similar to that of a red blood cell. Alternatively, the gel 10 may have a disk shape without dents indicated by the dashed lines.

Hereinafter, the method for producing a gel according to an embodiment of the invention will be described.

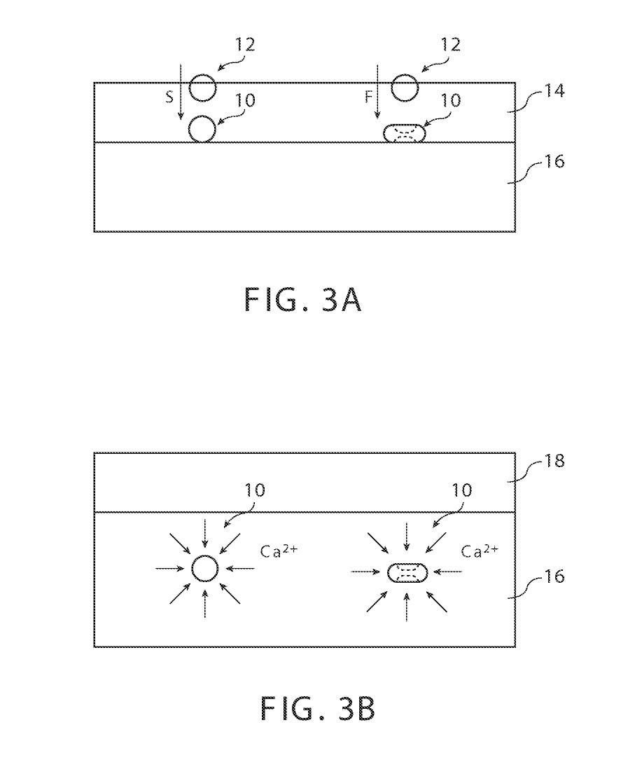

FIG. 2 is a flow chart showing the method for producing a gel according to this embodiment. FIGS. 3A and 3B are schematic process charts showing the method for producing a gel according to this embodiment. As shown in FIG. 2, the method for producing a gel according to this embodiment includes a liquid droplet ejection step (S10) and a size control step (S20). FIGS. 3A and 3B show the outlines of the steps, respectively.

As shown in FIG. 3A, the liquid droplet ejection step (S10) is performed. Specifically, a first solution (first liquid) 12 is ejected in the form of a droplet to a second solution (second liquid) 16 stored in a container 14 to allow the first solution 12 and the second solution 16 to react with each other, thereby producing the gel 10.

Here, FIGS. 3A and 3B each show a case S where a rate of ejecting the first solution 12 in the form of a droplet is slow (on the left side of the drawings) and a case F where the rate is fast (on the right side of the drawings).

The first solution 12 may be an aqueous solution of an alginate. For example, an aqueous solution of sodium alginate can be used. Further, an aqueous solution of fibrinogen, boric acid, or the like can be used.

The second solution 16 may be an aqueous solution of an alkaline earth metal salt. For example, an aqueous solution of calcium chloride, magnesium chloride, barium chloride, or the like can be used. Further, an aqueous solution of thrombin and a calcium salt such as calcium chloride, an aqueous solution of polyvinyl alcohol, or the like can be used.

For example, an aqueous solution of sodium alginate may be used as the first solution 12, and an aqueous solution of calcium chloride may be used as the second solution 16. When the aqueous solution of sodium alginate is ejected to the aqueous solution of calcium chloride, sodium alginate and calcium chloride chemically react with each other to produce the gel 10 composed of calcium alginate.

Then, as shown in FIG. 3B, the size control step (S20) is performed. Specifically, the gel 10 may be immersed in the aqueous solution of calcium chloride which is the second solution 16 stored in the container 18, and the size of the gel 10 is controlled. By doing this, the gelation of the gel 10 composed of calcium alginate is further induced by a divalent metal ion. The gelation proceeds by replacing a sodium ion (Na⁺) of sodium alginate remaining in the gel 10 with a calcium ion (Ca²⁺) which is a divalent metal ion. At this time, the sodium ion (Na⁺) is monovalent and the calcium ion (Ca²⁺) is divalent. Therefore, two sodium ions (Na⁺) are replaced by one calcium ion (Ca²⁺)

Sodium alginate remaining in a center region of the gel 10 is allowed to gelate as follows. Two sodium ions (Na⁺) are released from two sodium alginate molecules, and replaced by one calcium ion (Ca²⁺) which is a divalent metal ion, whereby crosslinking condensation occurs such that the two sodium alginate molecules are crosslinked through the calcium ion to cause gelation. By doing this, the size of the gel 10 is condensed, in other words, decreased.

Here, the illustration and description have been made such that the second solution 16 is stored in the container 18, however, the invention is not limited thereto, and as shown in FIG. 3A, in the liquid droplet ejection step (S10), the second solution 16 may be stored in the container 14.

When crosslinking condensation occurs to cause gelation, the gel 10 tries to maintain its shape before being immersed in the aqueous solution of calcium chloride, which is the second solution 16, due to the surface tension thereof. Therefore, as shown by the arrows in FIG. 3B, calcium ions (Ca²⁺) uniformly penetrate into a center region from the surface of the gel 10 and replace sodium ions (Na⁺).

In this manner, the gelation proceeds and the gel 10 is uniformly condensed. That is, in the case where the gel 10 has a spherical shape, the gel 10 is uniformly condensed while keeping the spherical shape. In the case where the gel 10 has a disk shape with dents or in the case where the gel 10 does not have a disk shape without dents as indicated by the dashed lines in the center, the gel 10 is uniformly condensed while keeping such a shape.

By controlling a time T for which the gel 10 is immersed in the aqueous solution of calcium chloride, which is the second solution 16, the degree of condensation of the size of the gel 10 can be regulated to thereby form the gel 10 with a desired size.

In this example, the aqueous solution of sodium alginate is ejected by an inkjet method. However, the strength of the gel may not be sufficient in some cases. Further, when water is serving as a main solvent in the aqueous solution, the water is liable to dry. As a result, the performance of intermittent ejection of the aqueous solution may be lowered or a nozzle may be clogged in some cases. Therefore, in order to form a harder gel and to prevent a nozzle from drying, a composition of the aqueous solution of sodium alginate for use in the inkjet method was studied by focusing on a polyhydric alcohol (glycerin) as a material which is a high-boiling solvent, has hygroscopicity, and is widely used.

The first solution 12 may be a gel forming solution composed of an aqueous solution containing an alcohol-based solvent. The alcohol-based solvent is contained therein in an amount of 4% or more by weight and less than 70% by weight of the total amount of the first solution 12. The alcohol-based solvent is at least one solvent selected, for example, from a group consisting of polyhydric alcohols. According to this configuration, by using one solvent selected from the group consisting of polyhydric alcohols, the alcohol-based solvent can be easily used.

The alcohol-based solvent may be contained therein in an amount of 40% or more by weight and less than 60% by weight of the total amount of the first solution 12. According to this configuration, by incorporating a specific alcohol-based solvent in the first solution 12 in a predetermined amount, a gel having a desired strength can be formed.

### Example

When alginate gel beads were produced, glycerin, which is a common polyhydric alcohol and is used also in pharmaceutical products and detergents, was added to the aqueous solution of sodium alginate. The evaluation was performed with respect to the following two items. One is a strength of the resulting gel, which was examined as follows. An aqueous solution of sodium alginate, in which an amount of glycerin added was changed, was allowed to gelate in the presence of calcium ions, and 15 μl of the resulting gel was examined for strength. The other item evaluated is a relationship between the amount of glycerin added and the performance of intermittent ejection, which was examined by adding glycerin to the aqueous solution of sodium alginate in different amounts.

The strength of the gel was examined in the following manner. First, the gel was placed on a scale such as an electronic balance. Subsequently, a load was applied to the gel on the scale in the direction from the opposite side of the scale to the scale using a finger or the like to crush the gel. The strength of the gel was determined from the graduation of the scale at this time.

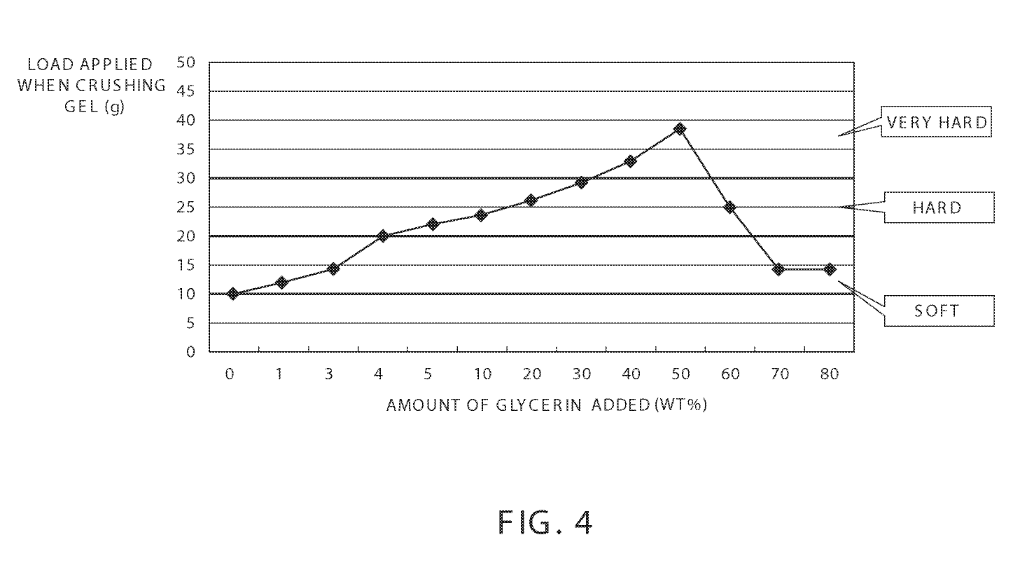

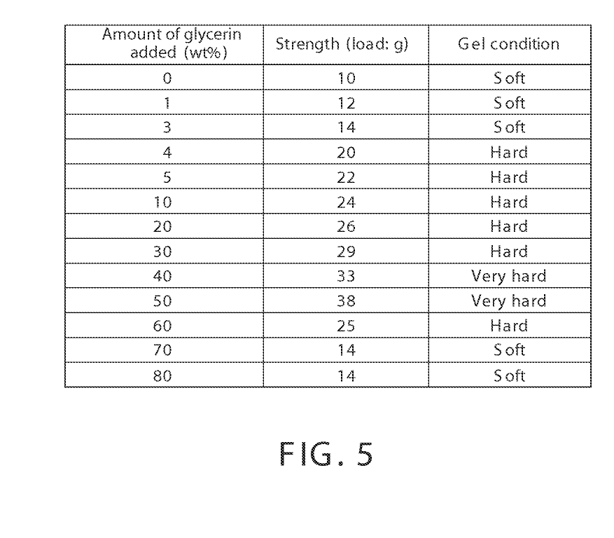

FIG. 4 is a graph showing a relationship between a gel strength and the amount of glycerin added to the gel forming solution according to this embodiment. FIG. 5 is a table showing a relationship between a strength (load) or a gel condition and the amount of glycerin added according to this embodiment.

The evaluation criteria for the gel condition are as follows: “soft” represents the condition of a gel which has a spherical shape and is soft; “hard” represents the condition of a gel which has a spherical shape and is hard to such an extent that the gel does not lose its shape, and “very hard” represents the condition of a gel which has a spherical shape and is rigid.

As shown in FIG. 4, the strength of the formed gel is increased within a certain range by adding glycerin. As for the relationship between the strength (strength (load)) of the resulting gel or the condition of the gel and the amount of glycerin added according to this embodiment, FIG. 5 shows that a load applied when crushing the formed gel increases within a certain range by adding glycerin. In other words, the gel condition is changed by adding glycerin.

Specifically, when the amount of glycerin added was 0% or more by weight and 3% or less by weight (e.g., between 0% and 3% by weight), the strength of the gel was from 10 to 14 g and the gel condition was soft. When the amount of glycerin added was 4% or more by weight and 30% or less by weight (e.g., between 4% and 30% by weight), the strength of the gel was from 20 to 29 g and the gel condition was hard. When the amount of glycerin added was 40% or more by weight and less than 60% by weight (e.g., between 40% and 50% by weight), the strength of the gel was from 33 to 38 g and the gel condition was very hard. When the amount of glycerin added was 60% by weight, the strength of the gel was 25 g and the gel condition was hard. When the amount of glycerin added was 70% or more by weight and 80% or less by weight (e.g., between 70% and 80% by weight), the strength of the gel was 14 g and the gel condition was soft. When the formed gel is soft, the shape of the gel is easily changed and can be controlled. On the other hand, it was found that when glycerin is added in a large amount, the gel strength is decreased.

According to this embodiment, by adjusting the amount of a specific alcohol-based solvent in the aqueous solution, a gel having a desired strength can be formed.

According to this embodiment, the addition of a polyhydric alcohol increases the gel strength. The concentration of glycerin for forming a hard gel is 4% or more by weight and less than 70% by weight, and the concentration of glycerin for forming a very hard gel is 40% or more by weight and less than 60% by weight.

The alcohol-based solvent according to this embodiment may be a low-volatile solvent, and specifically, an alcohol having a boiling point of from 150 to 350° C., from 200 to 350° C., or from 250 to 350° C. can be used. Among these, at least one solvent selected from the group consisting of polyhydric alcohols is used in one embodiment.

The gel forming solution according to this embodiment preferably contains a polyhydric alcohol as an organic solvent. The polyhydric alcohol prevents a nozzle from clogging when an inkjet head is not used for a given time. Thus, clogged nozzles can be avoided during intermittent ejection of the solution from the nozzles.

Specific examples of the polyhydric alcohol include dihydric alcohols such as triethylene glycol, 3-methyl-1,5-pentanediol, 1,6-hexanediol, hexylene glycol, methylcyclohexanol, 2,4,7,9-tetramethyl-5-desine-4,7-diol, and octane diol; and trihydric alcohols such as 1,2,6-hexanetriol.

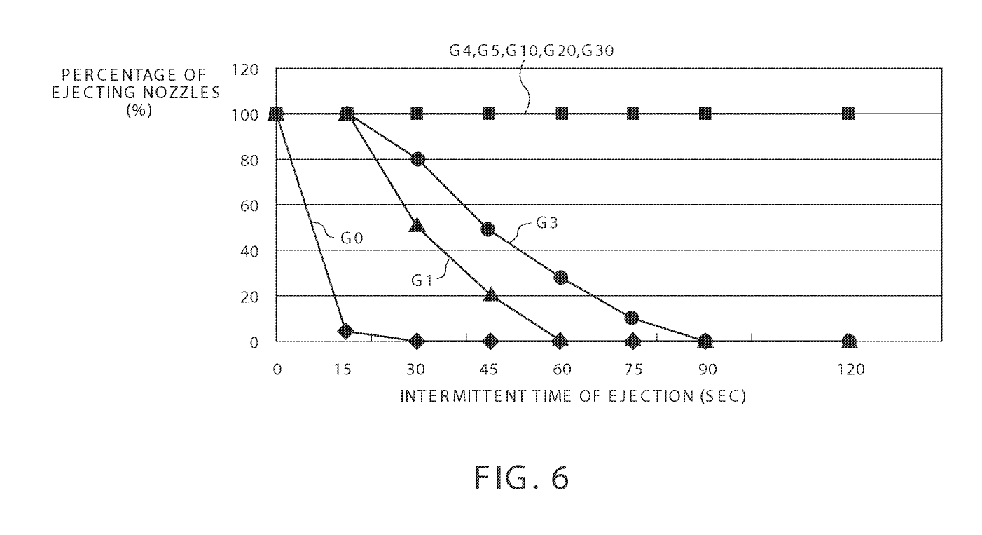

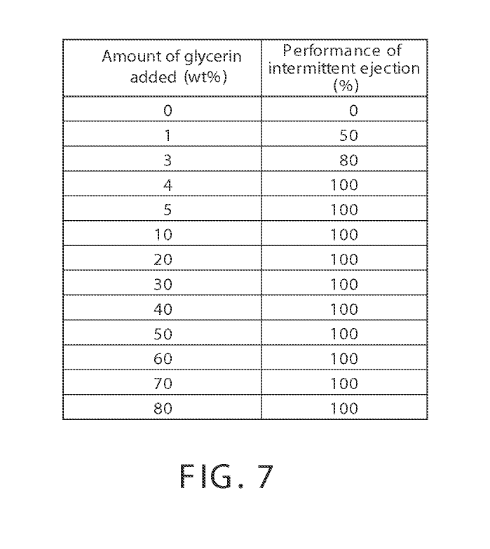

FIG. 6 is a graph showing a relationship between an intermittent time of ejection (e.g., time between ejections from a nozzle or from the nozzles) and a percentage of ejecting nozzles in response to a change in an amount of glycerin added to the gel forming solution according to this embodiment. FIG. 7 is a table showing a relationship between an amount of glycerin added to the gel forming solution according to this embodiment and performance of intermittent ejection. The graph in FIG. 6 is obtained by plotting the percentage of ejecting nozzles against the intermittent time of ejection and shows how the percentage of ejecting nozzles changes based on the initial value (100% ejection) in response to the amount of glycerin added. The table in FIG. 7 shows the percentage of the number of nozzles which could eject the solution again 30 seconds after finishing the previous ejection to the total number of nozzles when the intermittent time of ejection was set to 30 seconds. In other words, the solution is ejected from the nozzles. Thirty seconds later (an example of an intermittent time of ejection), by way of example only, the solution is ejected again and FIG. 7 illustrates a relationship between the amount of glycerin added to the gel forming solution and the percentage of nozzles (nozzles that can eject/total number of nozzles) that can eject the solution after thirty seconds.

As for the relationship between the intermittent time of ejection and the percentage of ejecting nozzles in response to a change in an amount of glycerin added according to this embodiment, as shown in FIG. 6, the percentage of ejecting nozzles is increased by increasing the amount of glycerin added to the gel forming solution. Thus, the addition of glycerin can prevent nozzles from clogging during the time between ejections or during the intermittent time of ejection.

Specifically, when the amount of glycerin added to the gel forming solution was 0% by weight, as shown in the line G0, the percentage of ejecting nozzles at a 30-second intermittent ejection was 0%. When the amount of glycerin added to the gel forming solution was 1% by weight, as shown in line G1 in FIG. 7, the percentage of ejecting nozzles at a 30-second intermittent ejection was 50%. When the amount of glycerin added to the gel forming solution was 3% by weight, as shown in line G3, the percentage of ejecting nozzles at a 30-second intermittent ejection was 80%. When the amount of glycerin added to the gel forming solution was 4% or more by weight, as shown in the lines G4, G5, G10, G20, and G30, the percentage of ejecting nozzles at a 30-second intermittent ejection time was 100%.

An organic solvent other than alcohol-based solvents has a low water retaining property and the water content reaches a saturation level in a short time. Therefore, water is easily released. On the other hand, an alcohol-based solvent has a high saturated water content and it takes a long time for the water content to reach a saturation level. Therefore, water is hardly released when the environment changes. Accordingly, by using the former organic solvent and an alcohol-based solvent in combination, the water retaining property of the gel forming solution can be increased. On the other hand, from the viewpoint of miscibility with the gel forming solution, a solvent of the same type as a solvent contained in the gel forming solution is preferably contained. In this manner, by using the former organic solvent and an alcohol-based solvent in combination, a gel forming solution having excellent miscibility with the gel forming solution can be obtained.

As for the relationship between the amount of glycerin added according to this embodiment and the performance of intermittent ejection, as shown in FIG. 7, increasing the addition amount of glycerin, improves the performance of intermittent ejection. In order to maintain favorable performance of intermittent ejection, glycerin is preferably added in an amount of 4% or more by weight.

Specifically, when the amount of glycerin added was 0% by weight, the performance of intermittent ejection was 0%. When the amount of glycerin added was 1% by weight, the performance of intermittent ejection was 50%. When the amount of glycerin added was 3% by weight, the performance of intermittent ejection was 80%. When the amount of glycerin added was 4% or more by weight, the performance of intermittent ejection was 100%.

According to this embodiment, the addition of a polyhydric alcohol improves the performance of intermittent ejection. The concentration of glycerin for achieving the performance of intermittent ejection is 4% or more by weight.

According to this embodiment, the alcohol-based solvent exhibits a high water retaining property. As a result, the saturated water content of the gel forming solution can be increased so as to cause less water to be released even if the environment changes. Therefore, by filling an ink flow path or a nozzle of an inkjet head with this gel forming solution, problems such as failure of the gel forming solution to eject and deterioration of the gel forming solution can be solved when the solution is ejected from the inkjet head and/or when the solution is ejected intermittently. As a result, by employing an inkjet method, mass production of a gel can be achieved and a gel can be produced at a desired timing. Therefore, the production cost of the gel can be suppressed.

As the additive, not only glycerin, but also a polyhydric alcohol such as ethylene glycol can be used.

The gel formed by the method for producing a gel described above can be preferably used in a wide range of fields. Pharmaceutical products, cosmetic products, agricultural chemicals, biomaterial analyses, and foods, and also regenerative medicine and the like are examples of fields where the gel can be used.

Modifications, improvements, and the like within a range in which at least a part of the problem described above can be solved are included in the embodiments described above.

For example, a time T for which the gel 10 is immersed in the second solution 16 can be used as the condition for condensing the gel 10 in the size control step (S20). However, the invention is not limited thereto, and it is possible to use a temperature of the second solution 16, the concentration of a solute in the second solution 16, or the divalent cation concentration of the second solution 16 as the condition for condensation.

The description has been made of a case where a calcium ion (Ca²⁺) is used as the divalent metal ion. However, the divalent metal ion may be an alkaline earth metal ion such as a magnesium ion (Mg²⁺) or a barium ion (Ba²⁺). In this case, magnesium chloride or barium chloride can be used in place of calcium chloride.

In the embodiment described above, the description has been made of a case where the second solution 16 is stored at rest in the container 14 or 18 without allowing the solution to flow. However, the invention is not limited thereto and may be configured such that the second solution 16 is allowed to flow into the container 14 or 18. In this case, in the same manner as the embodiment described above, in the size control step (S20), the effect described above can be obtained by controlling a time T for which the gel 10 is immersed in the second solution 16, the temperature of the second solution 16, the concentration of a solute in the second solution 16, and/or the divalent cation concentration of the second solution 16.

In the embodiment described above, a description has been made of a case where in order to obtain the alginate gel 10, an aqueous solution of sodium alginate is used as the first solution 12 and an aqueous solution of calcium chloride is used as the second solution 16. In another embodiment, a method in which an aqueous solution of potassium alginate is used as the first solution 12 and an aqueous solution of barium chloride is used as the second solution 16, or the like may be employed in order to obtain the alginate gel 10. Any method can be employed as long as the second solution 16 reacts with the first solution 12 containing a gel forming material to cause gelation.

It is also possible to incorporate a desired substance in the gel 10. For example, in an aqueous solution of sodium alginate, a hardening agent, a medicinal agent, an enzyme, a cell, a pigment, a catalyst, a nanoparticle, a fluorescent particle, and/or the like may be incorporated. The resulting solution is ejected in the form of a droplet. By doing this, the gel 10 can be obtained that includes a hardening agent, a medicinal agent, an enzyme, a cell, a pigment, a catalyst, a nanoparticle, a fluorescent particle, and/or the like incorporated therein.

For example, the gel 10 having a hardening agent or a medicinal agent incorporated therein enables the usage thereof such that when an external pressure or the like is applied to the gel 10, the hardening agent or medicinal agent comes out of the gel 10 and a hardening action or a medicinal action starts. Further, by forming a minute liquid droplet, it is possible to produce the gel 10 capable of exhibiting a hardening action or a medicinal action even in a narrow area. In particular, by producing the minute gel 10 having a hardening agent, a medicinal agent, or other agent incorporated therein as a dental material, it is possible to deliver an adequate amount of the hardening agent, medicinal agent, or other agent to a narrow area such as a tooth crown. This can avoid excessive usage of the hardening agent, medicinal agent, or other agent to prevent waste of such an agent, and can also reduce the cost of dental treatment. Also, for a pharmaceutical product, a cosmetic product, an agricultural chemical, a biomaterial analysis, a food, or the like, the amount of a gel forming material and the amount of a material to be incorporated into the gel can be reduced to a necessary minimum. As a result, the cost of the materials can be lowered. The size of the liquid droplet and the concentration of each liquid are not limited to those described in the embodiments.

What is claimed is:

1. A gel forming solution, comprising an alcohol-based solvent, wherein
the alcohol-based solvent is contained in the gel forming solution in an amount of 4% or more by weight and less than 70% by weight of a total amount of the gel forming solution.

2. The gel forming solution according to claim 1, wherein the alcohol-based solvent is at least one solvent selected from a group consisting of polyhydric alcohols.

3. The gel forming solution according to claim 1, wherein the alcohol-based solvent is contained therein in an amount of 40% or more by weight or more and less than 60% by weight of the total amount of the gel forming solution.

4. A method for producing a gel, comprising:
ejecting a first liquid in a form of a droplet to a second liquid to allow the first liquid and the second liquid to react with each other to produce a gel; wherein
the first liquid is a gel forming solution containing an alcohol-based solvent in an amount of 4% or more by weight and less than 70% by weight of a total amount of the gel forming solution.

5. The method for to the gel forming solution producing a gel according to claim 4, wherein in the ejecting, at least the first liquid is ejected in the form of a droplet.

6. A method for controlling a strength of a gel, the method comprising:
adding an alcohol-based solvent to a first liquid, wherein an amount of the alcohol-based solvent in the first liquid determines the strength of the gel; and
mixing the first liquid with the second liquid to produce the gel.

7. The method of claim 6, wherein mixing the first liquid with the second liquid comprises ejecting the first liquid in a form of a droplet.

8. The method of claim 7, wherein the amount of alcohol-based solvent results in the strength being:
soft when the amount of alcohol-based solvent is 3% or less by weight of the first liquid;
hard when the amount of alcohol-based solvent is 4% or more by weight and less than 40% by weight of the first liquid; and
very hard when the amount of alcohol-based solvent is 40% or more by weight and less than 60% by weight of the first liquid.

9. The method of claim 7, wherein the first liquid is one of an aqueous solution of sodium alginate or potassium alginate and the second liquid is one of an aqueous solution of calcium chloride or barium chloride.

10. The method of claim 6, further comprising incorporating one or a hardening agent, a medicinal agent, an enzyme, a cell, a pigment, a catalyst, a nanoparticle, or a fluorescent particle in the gel.

11. The method of claim 7, further comprising condensing the gel to a desired size.

12. The method of claim 11, further comprising condensing the gel to the desired size by controlling one or more of a time for which the gel is immersed in the second liquid, a temperature of the second liquid, a concentration of a solute in the second liquid, and/or a divalent cation concentration of the second liquid.

13. The method of claim 7, wherein the amount of alcohol-based solvent is 4% or more by weight and 70% or less by weight.
